(19) United States
(12) Patent Application Publication
(10) Pub. No.: US 2010/0255559 A1
(43) Pub. Date: Oct. 7, 2010

(54) APPARATUS AND PROCESS FOR TREATING AN AQUEOUS SOLUTION CONTAINING BIOLOGICAL CONTAMINANTS

(75) Inventors: John L. Burba, III, Parker, CO (US); Tim L. Oriard, Issaquah, WA (US)

Correspondence Address: SHERIDAN ROSS PC, 1560 BROADWAY, SUITE 1200, DENVER, CO, 80202, US

(73) Assignee: MOLYCORP MINERALS, LLC, Greenwood Village, CO (US)

(21) Appl. No.: 12/814,032

(22) Filed: Jun. 11, 2010

Related U.S. Application Data
(62) Division of application No. 11/931,616, filed on Oct. 31, 2007.

(51) Int. Cl. C12N 7/00; C12N 1/20; C12N 1/14; C12N 1/10

(52) U.S. Cl. 435/235.1; 435/252.1; 435/254.1; 435/257.1; 435/258.1

(57) ABSTRACT

Process, apparatus and article for treating an aqueous solution containing biological contaminants. The process includes contacting an aqueous solution containing a biological contaminant with an aggregate composition comprising an insoluble rare earth-containing compound to form a solution depleted of active biological contaminants. The aggregate includes more than 10.01% by weight of the insoluble rare earth-containing compound. The insoluble rare earth-containing compound can include one or more of cerium, lanthanum, or praseodymium. A suitable insoluble cerium-containing compound can be derived from a cerium carbonate, a cerium oxalate or a cerium salt. The composition can consist essentially of cerium oxides, and optionally, a binder and/or flow aid. The aggregate includes no more than two elements selected from the group consisting of yttrium, scandium, and europium when the aggregate is to be sintered. Although intended for a variety of fluid treatment applications, such applications specifically include removing or deactivating biological contaminants in water.

### FIELD OF THE INVENTION

The invention relates generally to the field of fluid and solution treatment, and primarily to processes and apparatuses for treating aqueous solutions. In its more particular aspects, the invention relates to processes, apparatuses and articles useful for removing or deactivating bacteria and viruses in aqueous solutions.

### BACKGROUND OF THE INVENTION

The purification and filtration of water and other aqueous solutions is necessary for many applications such as the provision of safe or potable drinking water, industrial processes requiring purified feeds, the handling of waste streams, and environments in which fluids must be treated prior to re-circulation such as found on ships, aircraft and spacecraft. In recent years, the increased need for purified solutions has lead to the development of numerous filtration products that purport to remove small particles, allergens, microorganisms, biotoxins, pesticides, and toxic metals such as lead, mercury, and arsenic.

Known methods for purifying aqueous solutions include reverse osmosis, distillation, ion-exchange, chemical adsorption, coagulation, flocculation, and filtering or retention. In some applications a combination of techniques is required in order to purify such solutions. Examples of this practice include the use of mixed ion-exchange resins that remove both negative and positively charged chemical species and oxidation/filtration methods where oxidizers are used to generate particulate matter that may be subsequently filtered. These purification practices can be costly, energy inefficient and require significant technical know-how and sophistication to implement on both large and small scales. As a result, many advanced fluid purification technologies have had limited application beyond municipal or industrial applications.

Some contaminants can be filtered through the use of membranes or layers of granular materials. For example, biological contaminants such as bacteria and fungi can be removed from fluids through ultrafiltration, but viruses are generally too small for filtration to be an effective means of purification. Because filtration is only effective at removing some biological contaminants, treatment with chemical additives tends to be the method of choice for purifying aqueous solutions containing diverse biological contaminants. Examples of chemical additives include oxidizing agents, flocculating agents, and precipitation agents. By way of example, biological contaminants such as bacteria, viruses and fungi have typically been removed from solution or deactivated by the action of strong oxidizing agents such as chlorine, hydrogen peroxide, ozone or quaternary amine salts. However, the use of chemical additive(s) can be costly and require special handling, transport, and storage, rendering them less desirable for many applications. Moreover, chemical treatment methods require careful administration and monitoring of the treated solutions. For example, where the application is a potable water system, chemical tablets or liquids are being added to water that will ultimately be consumed. In administering such chemicals, one must insure that appropriate conditions exist for the chemicals to thoroughly treat the water. Mistakes such as adding too much or too little of a chemical agent can lead to the failure to adequately treat the biological contaminants or result in unnecessary exposure to corrosive chemicals.

As a result, simplified means for removing biological contaminants from aqueous solutions is desired.

### SUMMARY OF THE INVENTION

In one embodiment, the invention provides a process for treating an aqueous solution containing a biological contaminant. The process includes contacting an aqueous solution containing biological contaminants with an aggregate composition comprising an insoluble rare earth-containing compound to form a solution depleted of active biological contaminants.

The aqueous solution can contact the aggregate composition by one or more of flowing the aqueous solution through the aggregate composition, distributing the aggregate composition over the surface of the aqueous solution, and submerging a fluid permeable container enclosing the aggregate composition into the aqueous solution. The aggregate composition can be disposed in a container and the aqueous solution can flow through the composition under the influence of one or more of gravity or pressure. The composition can be disposed in one or more of a fixed bed, fluidized bed, stirred tank and filter. The composition can also be disposed in a removable container and the process can include the step of intermittently replacing the removable container.

The aqueous solution contacts the composition at a temperature above the triple point for the aqueous solution. In some cases, the aqueous solution contacts the composition at a temperature less than about 100° C., and in other cases at a temperature less than about 80° C. In other cases, the aqueous solution contacts the composition at a temperature above about 100° C., at a pressure sufficient to maintain at least a portion of the aqueous solution in a liquid phase.

The process can optionally include one or more of the steps of separating the aqueous solution depleted of active biological contaminants from the aggregate composition, sensing the aqueous solution depleted of active biological contaminants, evaporating residual aqueous solution from the aggregate composition, intermittently replacing the aggregate composition, and sterilizing the aggregate composition after contacting the aqueous solution with the aggregate composition. Sterilizing the composition can be achieved by treating the aggregate composition with one or more of heat, radiation and a chemical agent. If the aqueous solution is to be treated with air, oxygen-enriched air, ozone or hydrogen peroxide for the purpose of oxidizing fungi and viruses that may be present in the solution, the solution is to be contacted with the aggregate composition prior to any such treatment.

The insoluble rare earth-containing compound can include one or more of cerium, lanthanum, or praseodymium amongst other rare earth-containing compounds. When the insoluble rare earth-containing compound comprises a cerium-containing compound, the cerium-containing compound can be derived from one or more of thermal decomposition of a cerium carbonate, decomposition of a cerium oxalate and precipitation of a cerium salt. The insoluble rare earth-containing compound can include a cerium oxide, and in some cases, the aggregate composition can consists essentially of one or more cerium oxides, and optionally, one or more of a binder and flow aid.

The aggregate composition will include more than 10.01% by weight of the insoluble rare earth-containing compound and can include more than 95% by weight of the insoluble rare earth-containing compound. The insoluble rare earth-containing compound can comprise particulates having a mean surface area of at least about 1 m²/g. When the insoluble rare earth-containing compound is in the form of a particulate, the particulate can have a mean particle size of at least about 1 nm. The aggregate composition can comprise aggregated particulates having a mean aggregate size of at least about 1 μm. When the aggregate composition has been sintered, it will include no more than two elements selected from the group consisting of yttrium, scandium, and europium.

In another embodiment, the invention provides an apparatus for treating an aqueous solution containing a biological contaminant. The apparatus includes a container having a fluid flow path for an aqueous solution and an aggregate composition disposed in the fluid flow path. The container can include one or more of a fixed bed, a fluidized bed or stirred tank and filter. In some cases, the container is adapted to be removed from the apparatus, such a container having an inlet and an outlet with each of the inlet and the outlet adapted to be sealed when removed from the apparatus. In other embodiments, the container includes a fluid permeable outer wall encapsulating the aggregate composition.

The apparatus can include a filter disposed in the fluid flow path downstream of the aggregate composition. The apparatus can optionally include one or more of a visual indicator for indicating when the aggregate composition should be replaced, a sensor for sensing an effluent flowing out of the container, and means for sterilizing the aggregate composition. Means for sterilizing the composition can include one or more of means for heating the aggregate composition, means for irradiating the aggregate composition and means for introducing a chemical agent into the fluid flow path.

The aggregate composition comprises an insoluble rare earth-containing compound for removing or deactivating biological contaminants in an aqueous solution. The aggregate composition will include more than 10.01% by weight of the insoluble rare earth-containing compound. The insoluble rare earth-containing compound can include one or more of cerium, lanthanum, or praseodymium amongst other rare earth-containing compounds. When the insoluble rare earth-containing compound comprises a cerium-containing compound, the cerium-containing compound can be derived from one or more of thermal decomposition of a cerium carbonate, decomposition of a cerium oxalate and precipitation of a cerium salt. The rare earth-containing compound can include a cerium oxide, and in some cases, the aggregate composition can consist essentially of one or more cerium oxides, and optionally, one or more of a binder and flow aid. When the insoluble rare earth-containing compound is in the form of a particulate, the particulate can have a mean particle size of at least about 1 nm. The insoluble rare earth-containing compound can comprise particulates having a mean surface area of at least about 1 m²/g.

The aggregate composition can include aggregated particulates having a mean aggregate size of at least about 1 μm. When the aggregate composition has been sintered, it will include no more than two elements selected from the group consisting of yttrium, scandium, and europium.

In another embodiment, the invention provides an article comprising a container having one or more walls defining an interior space and a flowable aggregate composition disposed in the interior space. The container bears instructions for use of the aggregate composition to treat an aqueous solution containing a biological contaminant.

The aggregate composition will include more than 10.01% by weight of the insoluble rare earth-containing compound. The insoluble rare earth-containing compound can include one or more of cerium, lanthanum, or praseodymium amongst other rare earth-containing compounds. When the insoluble rare earth-containing compound comprises a cerium-containing compound, the cerium-containing compound can be derived from one or more of thermal decomposition of a cerium carbonate, decomposition of a cerium oxalate and precipitation of a cerium salt. The insoluble rare earth-containing compound can include a cerium oxide, and in some cases, the aggregate composition can consist essentially of one or more cerium oxides, and optionally, one or more of a binder and flow aid. When the insoluble rare earth-containing compound is in the form of a particulate, the particulate can have a mean particle size of at least about 1 nm. The insoluble rare earth-containing compound can comprise particulates having a mean surface area of at least about 1 m²/g.

The aggregate composition can comprise aggregated particulates having a mean aggregate size of at least about 1 μm. When the aggregate has been sintered, it will include no more than two elements selected from the group consisting of yttrium, scandium, and europium.

### DETAILED DESCRIPTION OF THE PREFERRED EMBODIMENTS

Illustrative embodiments of the invention are described below. In the interest of clarity, not all features of an actual embodiment are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover it will be appreciated that such a development effort might be complex and time-consuming, but would nevertheless be a routine undertaking for those of ordinary skill in the art having the benefit of this disclosure.

As used herein, “one or more of” and “at least one of” when used to preface several elements or classes of elements such as X, Y and Z or X₁—Xₙ, Y₁—Yₙ and Z₁-Zₙ, is intended to refer to a single element selected from X or Y or Z, a combination of elements selected from the same class (such as X₁ and X₂), as well as a combination of elements selected from two or more classes (such as Y₁ and Zₙ).

It will be understood that a process, apparatus or article as described herein can be used to treat an aqueous solution containing a biological contaminant, and in particular, to remove or deactivate a biological contaminant such as bacteria and/or viruses that may be found in such solutions. Examples of solutions that can be effectively treated include solutions in potable water systems, in waste water treatment systems, and feed, process or waste streams in various industrial processes among others. The described processes, apparatuses and articles can be used to remove biological contaminants from solutions having diverse volume and flow rate characteristics and can be applied in variety of fixed, mobile and portable applications. While portions of the disclosure herein describe the removal of biological contaminants from water, and in particular from potable water streams, such references are illustrative and are not to be construed as limiting.

The terminology “remove” or “removing” includes the sorption, precipitation, conversion or killing of pathogenic and other microorganisms, such as bacteria, viruses, fungi and protozoa that may be present in aqueous solutions. The term “deactivate” or “deactivation” includes rendering a microorganism non-pathogenic to humans or other animals such as for example by killing the microorganism. The described processes, apparatuses and articles are intended to remove or deactivate biological contaminants such that the treated solutions meet or exceed standards for water purity established by various organizations and/or agencies including, for example, the American Organization of Analytical Chemists (AOAC), the World Health Organization, and the United States Environmental Protection Agency (EPA). Advantageously, water treated by the described processes and apparatuses can meet such standards without the addition of further disinfecting agents, e.g., chlorine or bromine.

The terms “microbe”, “microorganism”, “biological contaminant”, and the like include bacteria, fungi, protozoa, viruses, algae and other biological entities and pathogenic species that can be found in aqueous solutions. Specific non-limiting examples of biological contaminants can include bacteria such as Escherichia coli, Streptococcus faecalis, Shigella spp, Leptospira, Legimella pneumophila, Yersinia enterocolitica, Staphylococcus aureus, Pseudomonas aeruginosa, Klebsiella terrigena, Bacillus anthracis, Vibrio cholerae, Salmonella typhi, viruses such as hepatitis A, noroviruses, rotaviruses, and enteroviruses, protozoa such as Entamoeba histolytica, Giardia, Cryptosporidium parvum, and others. Biological contaminants can also include various species such as fungi or algae, which although generally non-pathogenic in nature, are advantageously removed to improve the aesthetic properties of water. How such biological contaminants came to be present in the aqueous solution, either through natural occurrence or through intentional or unintentional contamination, is non-limiting of the invention.

In one embodiment of the invention, a process is provided for treating an aqueous solution containing a biological contaminant. The process includes contacting an aqueous solution containing a biological contaminant with an aggregate composition that comprises an insoluble rare earth-containing compound. As used herein, “insoluble” is intended to refer to materials that are insoluble in water, or at most, are sparingly soluble in water under standard conditions of temperature and pressure. Contact by and between the aqueous solution and the aggregate composition removes and/or deactivates the biological contaminant to yield a solution depleted of active biological contaminants.

The aggregate composition comprises more than 10.01% by weight of the insoluble rare earth-containing compound. The amount of insoluble rare earth-containing compound can constitute more than about 11%, more than about 12% or more than about 15% by weight of the aggregate composition. In some cases a higher concentrations of rare earth compounds may be desirable. Depending on the application, the composition can constitute at least about 20%, in other cases at least about 50%, in still others at least about 75%, and in yet still others more than 95%, by weight of an insoluble rare earth-containing compound.

The insoluble rare earth-containing compound can include one or more of the rear earths including lanthanum, cerium, praseodymium, neodymium, promethium, samarium, europium, gadolinium, terbium, dysprosium, holmium erbium, thulium, ytterbium and lutetium. In some embodiments, the insoluble rare-earth containing compound can comprise one or more of cerium, lanthanum, or praseodymium. Insoluble rare earth-containing compounds are available commercially and may be obtained from any source or through any process known to those skilled in the art. The aggregate composition need not include a single rare earth-containing compound but can include two or more insoluble rare earth-containing compounds. Such compounds can contain the same or different rare earth elements and can contain mixed valence or oxidation states. By way of example, when the insoluble rare earth-containing compound comprises cerium, the aggregate composition can comprise one or more cerium oxides such as CeO₂ (IV) and Ce₂O₃ (III).

In an embodiment where the insoluble rare earth-containing compound comprises a cerium-containing compound, the cerium-containing compound can be derived from precipitation of a cerium salt. In another embodiment, an insoluble cerium-containing compound can be derived from a cerium carbonate or a cerium oxalate. More specifically, an insoluble cerium-containing compound can be prepared by thermally decomposing a cerium carbonate or oxalate at a temperature between about 250° C. and about 350° C. in a furnace in the presence of air. The temperature and pressure conditions may be altered depending on the composition of the cerium-containing starting materials and the desired physical properties of the insoluble rare earth-containing compound. The thermal decomposition of cerium carbonate may be summarized as:

Ce₂(CO₃)₃+½O₂→2CeO₂+3CO₂

The product may be acid treated and washed to remove remaining carbonate. Thermal decomposition processes for producing cerium oxides having various features are described in U.S. Pat. No. 5,897,675 (specific surface areas), U.S. Pat. No. 5,994,260 (pores with uniform lamellar structure), U.S. Pat. No. 6,706,082 (specific particle size distribution), and U.S. Pat. No. 6,887,566 (spherical particles), and such descriptions are incorporated herein by reference. Cerium carbonate and materials containing cerium carbonate are commercially available and may be obtained from any source known to those skilled in the art.

In embodiments where the insoluble rare earth-containing compound comprises a cerium-containing compound, the insoluble cerium-containing compound can include a cerium oxide such as CeO₂. In a particular embodiment, the aggregate composition can consists essentially of one or more cerium oxides, and optionally, one or more of a binder and flow aid.

The insoluble rare earth-containing compound can be present in the aggregate composition in the form of one or more of a granule, crystal, crystallite, particle or other particulate, referred to generally herein as a “particulate.” The particulates of the insoluble rare earth-containing compounds can have a mean particle size of at least about 0.5 nm ranging up to about 1 μm or more. Specifically, such particulates can have a mean particle size of at least about 0.5 nm, in some cases greater than about 1 nm, in other cases, at least about 5 nm, and still other cases at least about 10 nm, and in yet still other cases at least about 25 nm. In other embodiments, the particulates can have mean particle sizes of at least about 100 nm, specifically at least about 250 nm, more specifically at least about 500 nm, and still more specifically at least about 1 μm.

To promote interaction of the rare earth-containing compound with a biological contaminant in solution, the aggregate composition can comprise aggregated particulates of the insoluble rare earth-containing compound having a mean surface area of at least about 5 m²/g. Depending upon the application, higher surface areas may be desired. Specifically, the aggregated particulates can have a surface area of at least about 70 m²/g, in other cases more than about 85 m²/g, in still other cases more than 115 m²/g, and in yet other cases more than about 160 m²/g. In addition, it is envisioned that particulates with higher surface areas will be effective in the described processes, apparatuses and articles. One skilled in the art will recognize that the surface area of the aggregate composition will impact the fluid dynamics of the aqueous solution. As a result, there may be a need to balance benefits that are derived from increased surface area with disadvantages such as pressure drop that may occur.

Optional components that are suitable for use in the aggregate composition can include one or more soluble rare earth-containing compounds, secondary biocidal agents, adsorbents, flow aids, binders, substrates, and the like. Such optional components may be included in the aggregate composition depending on the intended utility and/or the desired characteristics of the composition.

Optional components can include one or more soluble rare earth-containing compounds. Soluble rare earth-containing compounds can have different activities and effects. By way of example, some soluble rare earth-containing compounds have been recognized as having a bacteriostatic or antimicrobial effect. Cerium chloride, cerium nitrate, anhydrous ceric sulfate, and lanthanum chloride are described as having such activity in “The Bacteriostatic Activity of Cerium, Lanthanum, and Thallium”, Burkes et al., Journal of Bateriology, 54:417-24 (1947). Similarly, the use of soluble cerium salts such as cerium nitrates, cerous acetates, cerous sulfates, cerous halides and their derivatives, and cerous oxalates are described for use in burn treatments in U.S. Pat. No. 4,088,754, such descriptions being incorporated herein by reference. Other soluble rare earth-containing compounds, whether organic or inorganic in nature, may impart other desirable properties to the compositions and may optionally be used.

Secondary biocidal agents can optionally be included for targeting a particular biological contaminant or for enhancing the general capacity of the aggregate composition to remove biological contaminants. Materials that may be suitable for use as secondary biocidal agents include compounds that are known to possess activity for removing or deactivating biological contaminants, even when such materials are present in small quantities. Such materials include but are not limited to alkali metals, alkaline earth metals, transition metals, actinides, and derivatives and mixtures thereof. Specific non-limiting examples of secondary biocidal agents include elemental or compounds of silver, zinc, copper, iron, nickel, manganese, cobalt, chromium, calcium, magnesium, strontium, barium, boron, aluminum, gallium, thallium, silicon, germanium, tin, antimony, arsenic, lead, bismuth, scandium, titanium, vanadium, yttrium, zirconium, niobium, molybdenum, technetium, ruthenium, rhodium, palladium, cadmium, indium, hafnium, tantalum, tungsten, rhenium, osmium, iridium, platinum, gold, mercury, thallium, thorium, and the like. Derivatives of such agents can include acetates, ascorbates, benzoates, carbonates, carboxylates, citrates, halides, hydroxides, gluconates, lactates, nitrates, oxides, phosphates, propionates, salicylates, silicates, sulfates, sulfadiazines, and combinations thereof. When the aggregate composition optionally comprises a titanium-containing compound such as a titanium oxide, the weight ratio of the titanium-containing compound to the insoluble rare earth-containing compound is less than about 2:1. When the insoluble rare earth-containing compound has been sintered to form the aggregate composition, the composition will contain no more than two elements selected from the group consisting of yttrium, scandium, and europium. In an embodiment where the aggregate composition comprises an aluminum-containing compound, the weight ratio of the aluminum-containing compound to the insoluble rare earth-containing compound is less than about 10:1. In an embodiment that includes a secondary biocidal agent selected from the group consisting of transition metals, transition metal oxides and transition metal salts, the aggregate composition will comprise less than about 0.01% by weight of a mixture of silver and copper metal nanoparticles.

Other materials that may be suitable for use as secondary biocidal agents include organic agents such as quaternary ammonium salts as described in U.S. Pat. No. 6,780,332, and organosilicon compounds such as are described in U.S. Pat. No. 3,865,728. Other organic materials and their derivatives that are known to deactivate biological contaminants may also be used. By way of example, polyoxometalates are described in U.S. Pat. No. 6,723,349 as being effective at removing biological contaminants from fluids. This patent references M. T. in Heteropoly and Isopoly Oxometalates, Springer Verlag, 1983, and Chemical Reviews, vol. 98, No. 1, pp. 1-389, 1998, as describing examples of effective polyoxometalates. The descriptions of these organic biocidal agents in the noted references are incorporated herein by reference.

The aggregate composition may optionally comprise one or more flow aids. Flow aids are used in part to improve the fluid dynamics of a fluid over or through the aggregate composition, to prevent separation of components of the aggregate composition, prevent the settling of fines, and in some cases to hold the aggregate composition in place. Suitable flow aids can include both organic and inorganic materials. Inorganic flow aids can include ferric sulfate, ferric chloride, ferrous sulfate, aluminum sulfate, sodium aluminate, polyaluminum chloride, aluminum trichloride, silicas, diatomaceous earth and the like. Organic flow aids can include organic flocculents known in the art such as polyacrylamides (cationic, nonionic, and anionic), EPI-DMA's (epichlorohydrin-dimethylamines), DADMAC's (polydiallydimethyl-ammonium chlorides), dicyandiamide/formaldehyde polymers, dicyandiamide/amine polymers, natural guar, etc. When present, the flow aid can be mixed with the insoluble rare earth-containing compound and polymer binder during the formation of the aggregate composition. Alternatively, particulates of the aggregate composition and of the flow aid can be mixed to yield a physical mixture with the flow aid dispersed uniformly throughout the mixture. In yet another alternative, the flow aid can be disposed in one or more distinct layers upstream and downstream of the aggregate composition. When present, flow aids are generally used in low concentrations of less than about 20%, in some cases less than 15%, in other cases less than 10%, and in still other cases less than about 8% by weight of the aggregate composition.

Other optional components can include various inorganic agents including ion-exchange materials such as synthetic ion exchange resins, activated carbons, zeolites (synthetic or naturally occurring), clays such as bentonite, smectite, kaolin, dolomite, montmorillonite and their derivatives, metal silicate materials and minerals such as of the phosphate and oxide classes. In particular, mineral compositions containing high concentrations of calcium phosphates, aluminum silicates, iron oxides and/or manganese oxides with lower concentrations of calcium carbonates and calcium sulfates may be suitable. These materials may be calcined and processed by a number of methods to yield mixtures of varying compositions and properties.

A binder may optionally be included for forming an aggregate composition having desired size, structure, density, porosity and fluid properties. In addition to, or as an alternative to the use of a binder, a substrate may be included for providing support to the aggregate composition. Suitable binder and substrate materials can include any material that will bind and/or support the insoluble rare earth-containing compound under conditions of use. Such materials will generally be included in the aggregate composition in amounts ranging from about 0 wt % to about 90 wt %, based upon the total weight of the composition. Suitable materials can include organic and inorganic materials such as natural and synthetic polymers, ceramics, metals, carbons, minerals, and clays. One skilled in the art will recognize that the selection of a binder or substrate material will depend on such factors as the components to be aggregated, their properties and binding characteristics, desired characteristics of the final aggregate composition and its method of use among others.

Suitable polymer binders can include both naturally occurring and synthetic polymers, as well as synthetic modifications of naturally occurring polymers. In general, polymers melting between about 50° C. and about 500° C., more particularly, between about 75° C. and about 350° C., even more particularly between about 80° C. and about 200° C., are suitable for use in aggregating the components of the composition. Non-limiting examples can include polyolefins that soften or melt in the range from about 85° C. to about 180° C., polyamides that soften or melt in the range from about 200° C. to about 300° C., and fluorinated polymers that soften or melt in the range from about 300° C. to about 400° C.

Depending upon the desired properties of the composition, polymer binders can include one or more polymers generally categorized as thermosetting, thermoplastic, elastomer, or a combination thereof as well as cellulosic polymers and glasses. Suitable thermosetting polymers include, but are not limited to, polyurethanes, silicones, fluorosilicones, phenolic resins, melamine resins, melamine formaldehyde, and urea formaldehyde. Suitable thermoplastics can include, but are not limited to, nylons and other polyamides, polyethylenes, including LDPE, LLDPE, HDPE, and polyethylene copolymers with other polyolefins, polyvinylchlorides (both plasticized and unplasticized), fluorocarbon resins, such as polytetrafluoroethylene, polystyrenes, polypropylenes, cellulosic resins, such as cellulose acetate butyrates, acrylic resins, such as polyacrylates and polymethylmethacrylates, thermoplastic blends or grafts such as acrylonitrile-butadiene-styrenes or acrylonitrile-styrenes, polycarbonates, polyvinylacetates, ethylene vinyl acetates, polyvinyl alcohols, polyoxymethylene, polyformaldehyde, polyacetals, polyesters, such as polyethylene terephthalate, polyether ether ketone, and phenol-formaldehyde resins, such as resols and novolacs. Suitable elasomers can include, but are not limited to, natural and/or synthetic rubbers, like styrene-butadiene rubbers, neoprenes, nitrile rubber, butyl rubber, silicones, polyurethanes, alkylated chlorosulfonated polyethylene, polyolefins, chlorosulfonated polyethylenes, perfluoroelastomers, polychloroprene (neoprene), ethylene-propylene-diene terpolymers, chlorinated polyethylene, fluoroelastomers, and ZALAK™ (Dupont-Dow elastomer). Those of skill in the art will realize that some of the thermoplastics listed above can also be thermosets depending upon the degree of cross-linking, and that some of each may be elastomers depending upon their mechanical properties. The categorization used above is for ease of understanding and should not be regarded as limiting or controlling.

Cellulosic polymers can include naturally occurring cellulose such as cotton, paper and wood and chemical modifications of cellulose. In a specific embodiment, the insoluble rare earth-containing compound can be mixed paper pulp or otherwise combined with paper fibers to form a paper-based filter comprising the insoluble rare earth-containing compound.

Polymer binders can also include glass materials such as glass fibers, beads and mats. Glass solids may be mixed with particulates of an insoluble rare earth-containing compound and heated until the solids begin to soften or become tacky so that the insoluble rare earth-containing compound adheres to the glass. Similarly, extruded or spun glass fibers may be coated with particles of the insoluble rare earth-containing compound while the glass is in a molten or partially molten state or with the use of adhesives. Alternatively, the glass composition may be doped with the insoluble rare earth-containing compound during manufacture. Techniques for depositing or adhering insoluble rare earth-containing compounds to a substrate material are described in U.S. Pat. No. 7,252,694 and other references concerning glass polishing. For example, electro-deposition techniques and the use of metal adhesives are described in U.S. Pat. No. 6,319,108 as being useful in the glass polishing art. The descriptions of such techniques are incorporated herein by reference.

In some applications such as where a controlled release of the aggregate composition is desired, water-soluble glasses such as are described in U.S. Pat. Nos. 5,330,770, 6,143,318 and 6,881,766, may be an appropriate polymer binder. The descriptions of such glasses in the noted references are incorporated herein by reference. In other applications, materials that swell through fluid absorption including but not limited to polymers such as synthetically produced polyacrylic acids, and polyacrylamides and naturally-occurring organic polymers such as cellulose derivatives may also be used. Biodegradable polymers such as polyethylene glycols, polylactic acids, polyvinylalcohols, co-polylactideglycolides, and the like may also be used as the polymer binder.

Minerals and clays such as bentonite, smectite, kaolin, dolomite, montmorillonite and their derivatives may also serve as suitable binder or substrate materials.

Where it is desirable to regenerate the aggregate composition through sterilization, the selected binder or substrate material should be stable under sterilization conditions and should be otherwise compatible with the sterilization method. Specific non-limiting examples of polymeric binders that are suitable for sterilization methods that involve exposure to high temperatures include cellulose nitrate, polyethersulfone, nylon, polypropylene, polytetrafluoroethylene, and mixed cellulose esters. Compositions prepared with these binders can be autoclaved when the prepared according to known standards. Desirably, the aggregate composition should be stable to steam sterilization or autoclaving as well as to chemical sterilization through contact with oxidative or reductive chemical species, as a combination of sterilization methods may be required for efficient and effective regeneration. In an embodiment where sterilization includes the electrochemical generation of an oxidative or reductive chemical species, the electrical potential necessary to generate said species can be attained by using the composition as one of the electrodes. For example, a composition that contains a normally insulative polymeric binder can be rendered conductive through the inclusion of a sufficiently high level of conductive particles such as granular activated carbon, carbon black, or metallic particles. Alternatively, if the desired level of carbon or other particles is not sufficiently high to render an otherwise insulative polymer conductive, an intrinsically conductive polymer may included in the binder material. Various glasses such as microporous glass beads and fibers are particularly suited for use as a substrate or binder where the composition is to be periodically regenerated.

Other optional components of the aggregate composition can include additives, such as particle surface modification additives, coupling agents, plasticizers, fillers, expanding agents, fibers, antistatic agents, initiators, suspending agents, photosensitizers, lubricants, wetting agents, surfactants, pigments, dyes, UV stabilizers, and suspending agents. The amounts of these materials are selected to provide the properties desired. Such additives may be incorporated into a binder or substrate material, applied as a separate coating, held within the structure of the aggregate composition, or combinations of the above.

The aggregate composition can be formed though one or more of extrusion, molding, calcining, sintering, compaction, the use of a binder or substrate, adhesives and/or other techniques known in the art. It should be noted that neither a binder nor a substrate is required in order to form the aggregate composition although such components may be desired depending on the intended application. In embodiments where the aqueous solution is to be flowed through a bed of the aggregate composition, the composition can incorporate a polymer binder so that the resulting composition has both high surface area and a relatively open structure. Such an aggregate composition maintains elevated activity for removing or deactivating biological contaminants without imposing a substantial pressure drop on the treated solution. In embodiments where it is desired that the aggregate composition have higher surface areas, sintering is a less desirable technique for forming the aggregate composition. When the insoluble rare earth-containing compound has been sintered to form the aggregate composition, the composition will contain no more than two elements selected from the group consisting of yttrium, scandium, and europium.

In one embodiment, the aggregate composition can be produced by combining an insoluble rare earth-containing compound or a calcined aggregate of an insoluble rare earth-containing compound with a binder or substrate such as a polyolefin, cellulose acetate, acrylonitrile-butadiene-styrene, PTFE, a microporous glass or the like. The insoluble rare earth-containing compound, preferably in the form of a high surface area particulate, is mixed with the solid binder material. The mixture is then heated to a temperature, such as the glass transition temperature of the binder material, at which the solid binder material softens or becomes tacky. Depending on the temperature required to achieve a softened or tacky binder, the mixture may be heated at elevated pressure(s). The mixture is then allowed to cool so that mixture forms an aggregate with the insoluble rare earth-containing particulate adhered to the binder.

Where glass fibers or beads are used as a binder or substrate, the glass solids may be intimately mixed with particulates of an insoluble rare earth-containing compound and heated until the glass begins to soften or become tacky so that the insoluble rare earth-containing adheres to the glass upon cooling. Alternatively, the glass composition may be doped with the insoluble rare earth-containing compound during manufacture of the glass solids. Techniques for depositing or adhering insoluble rare earth-containing compounds to a substrate are described in U.S. Pat. No. 7,252,694 and other references concerning glass polishing. For example, electro-deposition techniques and the use of metal adhesives are described in U.S. Pat. No. 6,319,108 as being useful in the glass polishing art. The descriptions of such techniques are incorporated herein by reference.

Those familiar with the art of fluid treatment will understand that the components, physical dimensions and shape of the aggregate composition may be manipulated for different applications and that variations in these variables can alter flow rates, back-pressure, and the capacity of the composition to remove or deactivate biological contaminants. As a result, the size, form and shape of the aggregate composition can vary considerably depending on the method of use. Where the aqueous solution is to be flowed through the aggregate composition, such as in a column or other container, it desired that the aggregate composition have relatively open structure, with channels or pores that provide a high degree of fluid permeability and/or low density.

The aggregate composition can comprise aggregated particulates in granule, bead, powder, fiber or similar form. Such aggregated particulates can have a mean aggregate size of at least about 1 μm, specifically at least about 5 μm, more specifically at least about 10 μm, and still more specifically at least about 25 μm. In other embodiments, the aggregate will have a mean aggregate size of at least about 0.1 mm, specifically at least about 0.5 mm, more specifically at least about 1 mm, still more specifically at least about 2 mm, and yet still more specifically more than 5.0 mm. The aggregate composition can be crushed, chopped or milled and then sieved to obtain the desired particle size. Such aggregated particulates can be used in fixed or fluidized beds or reactors, stirred reactors or tanks, distributed in particulate filters, encapsulated or enclosed within membranes, mesh, screens, filters or other fluid permeable structures, deposited on filter substrates, and may further be formed into a desired shape such as a sheet, film, mat or monolith for various applications.

In addition, the aggregate composition can be incorporated into or coated onto a substrate. Suitable substrates can be formed from materials such as sintered ceramics, sintered metals, microporous carbon, glass and cellulosic fibers such as cotton, paper and wood. The structure of the substrate will vary depending upon the application but can include woven and non-wovens in the form of a porous membrane, filter or other fluid permeable structure. Substrates can also include porous and fluid permeable solids having a desired shape and physical dimensions. Such substrates can include mesh, screens, tubes, honeycombed structures, monoliths and blocks of various shapes including cylinders and toroids. In a particular embodiment, the aggregate composition and can be incorporated into or coated onto a filter block or monolith for use in cross-flow type filter.

The aggregate composition is used to treat an aqueous solution containing a biological contaminant by contacting the solution with the composition. Contact between the solution and the composition can be achieved by flowing the solution through the composition or by adding the composition to the solution, with or without mixing or agitation. If the aqueous solution is to be treated with air, oxygen-enriched air, ozone or hydrogen peroxide for the purpose of wet oxidizing fungi, viruses or other biological contaminants in the solution, then the aqueous solution is contacted with the aggregate composition prior to any such treatment with air, oxygen-enriched air, ozone or hydrogen peroxide. Contact with the aggregate composition is sufficient to remove or deactivate biological contaminants in the solution and the treatment of the aqueous solution with ozone or other agents for the purpose of wet oxidizing contaminants in solution is purely optional in nature.

In some embodiments, the aggregate composition is distributed over the surface of a solution and allowed to settle through the solution under the influence of gravity. Such an application is particularly useful for reducing biological contaminants in solutions found in evaporation tanks, municipal water treatment systems, fountains, ponds, lakes and other natural or man-made bodies of water. In such embodiments, it is preferred but not required that the composition be filtered or otherwise separated from the solution for disposal or regeneration and re-use.

In other embodiments, the aggregate composition can be introduced into the flow of the aqueous solution such as through a conduit, pipe or the like. Where it is desirable to separate the treated solution from the composition, the aggregate composition is introduced into the solution upstream of a filter where the composition can be separated and recovered from the solution. A particular example of such an embodiment can be found in a municipal water treatment operations where the composition is injected into the water treatment system upstream of a particulate filter bed.

In other embodiments, the aggregate composition can be disposed in a container and the solution directed to flow through the composition. The aqueous solution can flow through the composition under the influence of gravity, pressure or other means and with or without agitation or mixing. In still other embodiments, the container can comprise a fluid permeable outer wall encapsulating the aggregate composition so that the solution has multiple flow paths through the composition when submerged. Various fittings, connections, pumps, valves, manifolds and the like can be used to control the flow of the solution through the composition in a given container.

The aqueous solution contacts the aggregate composition at a temperature above the triple point for the solution. In some cases, the solution contacts the composition at a temperature less than about 100° C. and in other cases, contact occurs at a temperature above about 100° C., but at a pressure sufficient to maintain at least a portion of the aqueous solution in a liquid phase. The composition is effective at removing and deactivating biological contaminants at room temperatures. In other cases, the aqueous solution contacts the composition under supercritical conditions of temperature and pressure for the aqueous solution.

The pressure at which the aqueous solution contacts the aggregate composition can vary considerably depending on the application. For smaller volume applications where the contact is to occur within a smaller diameter column at a flow rates less than about 1.5 gpm, the pressure can range from 0 up to about 60 psig. In applications where larger containers and higher flow rates are employed, higher pressures may be required.

After contacting the aqueous solution, the aggregate composition may contain active and deactivated biological contaminants. As a result, it may be advantageous to sterilize the composition before re-use or disposal. Moreover, it may be desirable to sterilize the composition prior to contacting the aqueous solution to remove any contaminants that may be present before use. Sterilization processes can include thermal processes wherein the composition is exposed to elevated temperatures or pressures or both, radiation sterilization wherein the composition is subjected to elevated radiation levels, including processes using ultraviolet, infrared, microwave, and ionizing radiation, and chemical sterilization, wherein the composition is exposed to elevated levels of oxidants or reductants or other chemical species. Chemical species that may be used in chemical sterilization can include halogens, reactive oxygen species, formaldehyde, surfactants, metals and gases such as ethylene oxide, methyl bromide, beta-propiolactone, and propylene oxide. Combinations of these processes can also be used and it should further be recognized that such sterilization processes may be used on a sporadic or continuous basis while the composition is in use.

The process can optionally include the step of sensing the solution depleted of active biological contaminants so as to determine or calculate when it is appropriate to replace the composition. Sensing of the solution can be achieved through conventional means such as tagging and detecting the contaminants in the aqueous solution using fluorescent or radioactive materials, measuring flow rates, temperatures, pressures, sensing for the presence of fines, and sampling and conducting arrays. Techniques used in serology testing or analysis may also be suitable for sensing the solution depleted of active biological contaminants.

The process can optionally include separating the solution depleted of active biological contaminants from the composition. The composition can be separated from the solution by conventional liquid-solid separation techniques including, but not limited to, the use of filters, membranes, settling tanks, centrifuges, cyclones or the like. The separated solution depleted of active biological contaminants can then be directed to further processing, storage or use.

In another embodiment, the invention is directed to an apparatus for treating an aqueous solution containing a biological contaminant. The apparatus comprises a container having a fluid flow path and an aggregate composition as described herein disposed in the fluid flow path. Specifically, the aggregate composition comprises more than 10.01% by weight of the insoluble rare earth-containing compound and comprises no more than two elements selected from the group consisting of yttrium, scandium, and europium when the aggregate composition is sintered. Details of the aggregate composition are described elsewhere herein and are not repeated here.

The container can take a variety of forms including columns, various tanks and reactors, filters, filter beds, drums, cartridges, fluid permeable containers and the like. In some embodiments, the container will include one or more of a fixed bed, a fluidized bed, a stirred tank or reactor, or filter, within which the aqueous solution will contact the composition. The container can have a single pass through design with a designated fluid inlet and fluid outlet or can have fluid permeable outer wall enclosing or encapsulating the aggregate composition. Where it is desired that the container be flexible in nature, the fluid permeable outer wall can be made from woven or non-woven fabric of various water-insoluble materials so that the aqueous solution has multiple flow paths through the composition when submerged. Where a more rigid structure is preferred, the container can be manufactured from metals, plastics such as PVC or acrylic, or other insoluble materials that will maintain a desired shape under conditions of use.

The aqueous solution can flow through the composition and container under the influence of gravity, pressure or other means, with or without agitation or mixing. Various fittings, connections, pumps, valves, manifolds and the like can be used to control the flow of the solution into the container and through the composition.

The container can be adapted to be inserted into and removed from an apparatus or process stream to facilitate use and replacement of the composition. Such a container can have an inlet and outlet that are adapted to be sealed when removed from the apparatus or when otherwise not in use to enable the safe handling, transport and storage of the container and composition. Where the aggregate composition is to be periodically sterilized, the composition and container may be removed and sterilized as a unit, without the need to remove the composition from the container. In addition, such a container may also be constructed to provide long term storage or to serve as a disposal unit for biological contaminants removed from the solution.

The apparatus can include a filter for separating the treated solution from the composition. The filter can encapsulate the aggregate composition or be disposed downstream of the composition. Moreover, the filter can be a feature of the container for preventing the composition from flowing out of the container or be a feature of the apparatus disposed downstream of the container. The filter can include woven and non-woven fabrics, mesh, as well as fibers or particulates that are disposed in a mat, bed or layer that provides a fluid permeable barrier to the aggregate composition. Where the aggregate composition is disposed in a fixed bed, a suitable filter can will include a layer of diatomaceous earth disposed downstream of the composition within the container.

The apparatus may also optionally include one or more of a visual indicator for indicating when the composition should be replaced or regenerated, a sensor for sensing an effluent flowing out of the container, and means for sterilizing the composition. Means for sterilizing the composition can include one or more of means for heating the composition, means for irradiating the composition and means for introducing a chemical oxidation agent into the fluid flow path, such as are known in the art.

In yet another embodiment, the invention provides an article comprising a container having one or more walls defining an interior space and a flowable aggregate composition disposed in the interior space. As described in detail herein, the flowable aggregate composition comprises more than 10.01% by weight of an insoluble rare earth-containing compound and comprises no more than two elements selected from the group consisting of yttrium, scandium, and europium when the aggregate has been sintered. In addition, the container bears instructions for use of the aggregate composition to treat an aqueous solution containing a biological contaminant. In this particular embodiment, the container is a bag or other bulk product package in which the flowable aggregate composition may be marketed or sold to retailers, distributors or end use consumers. Such containers can take a variety of sizes, shapes, and forms, but are typically made from plastics or various fabrics. The container bears an instruction indicating that the contents of the container can be effectively used to treat aqueous solutions containing a biological contaminant for the purpose of removing or deactivating such a contaminant in the solution.

The following examples are provided to demonstrate particular embodiments of the present invention. It should be appreciated by those of skill in the art that the methods disclosed in the examples which follow merely represent exemplary embodiments of the present invention. However, those of skill in the art should, in light of the present disclosure, appreciate that many changes can be made in the specific embodiments described and still obtain a like or similar result without departing from the spirit and scope of the present invention.

### EXAMPLES

15 ml of CeO₂ obtained from Molycorp, Inc.'s Mountain Pass facility was placed in a ⅞″ inner diameter column.

600 ml of influent containing de-chlorinated water and 3.5×10⁴/ml of MS-2 was flowed through the bed of CeO₂ at flow rates of 6 ml/min, 10 ml/min and 20 ml/min. Serial dilutions and plating were performed within 5 minutes of sampling using the double agar layer method with E. Coli host and allowed to incubate for 24 hrs at 37° C.

The results of these samples are presented in Table 1.

TABLE 1

| Bed and | Influent | Effluent | Percent | |
|---|---|---|---|---|
| Flow Rate | Pop./ml | Pop/ml | reduction | Challenger |
| CeO₂ 6 ml/min | 3.5 × 10⁴ | 1 × 10⁰ | 99.99 | MS-2 |
| CeO₂ 10 ml/min | 3.5 × 10⁴ | 1 × 10⁰ | 99.99 | MS-2 |
| CeO₂ 20 ml/min | 3.5 × 10⁴ | 1 × 10⁰ | 99.99 | MS-2 |

The CeO₂ bed treated with the MS-2 containing solution was upflushed. A solution of about 600 ml of de-chlorinated water and 2.0×10⁶/ml of Klebsiella terrgena was prepared and directed through the column at flow rates of 10 ml/min, 40 ml/min and 80 ml/min. The Klebsiella was quantified using the Idexx Quantitray and allowing incubation for more than 24 hrs. at 37° C.

The results of these samples are presented in Table 2.

TABLE 2

| Bed and | Influent | Effluent | Percent | |
|---|---|---|---|---|
| Flow Rate | Pop./ml | Pop/ml | reduction | Challenger |
| CeO₂ 10 ml/min | 2.0 × 10⁶ | 1 × 10⁻² | 99.99 | Klebsiella |
| CeO₂ 40 ml/min | 2.0 × 10⁶ | 1 × 10⁻² | 99.99 | Klebsiella |
| CeO₂ 80 ml/min | 2.0 × 10⁶ | 1 × 10⁻² | 99.99 | Klebsiella |

The CeO₂ bed previously challenged with MS-2 and Klebsiella terrgena was then challenged with a second challenge of MS-2 at increased flow rates. A solution of about 1000 ml de-chlorinated water and 2.2×10⁵/ml of MS-2 was prepared and directed through the bed at flow rates of 80 ml/min, 120 ml/min and 200 ml/min. Serial dilutions and plating were performed within 5 minutes of sampling using the double agar layer method with E. Coli host and allowed to incubate for 24 hrs at 37° C.

The results of these samples are presented in Table 3.

TABLE 3

| Bed and | Influent | Effluent | Percent | |
|---|---|---|---|---|
| Flow Rate | Pop./ml | Pop/ml | reduction | Challenger |
| CeO₂ 80 ml/min | 2.2 × 10⁵ | 1 × 10⁰ | 99.99 | MS-2 |
| CeO₂ 120 ml/min | 2.2 × 10⁵ | 1.4 × 10² | 99.93 | MS-2 |
| CeO₂ 200 ml/min | 2.2 × 10⁵ | 5.6 × 10⁴ | 74.54 | MS-2 |

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

1-29. (canceled)

30. A composition, comprising:
a water insoluble rare earth-containing compound; and
a biological contaminant in contact with, and deactivated by, the water insoluble rare earth compound.

31. The composition of claim 30, wherein the rare earth-containing compound comprises no more than two elements selected from the group consisting of yttrium, scandium, and europium.

32. The composition of claim 30, wherein the rare earth in the rare earth compound is one or more of cerium, praseodymium, and lanthanum.

33. The composition of claim 30, wherein the biological contaminant comprises one or more of bacteria, fungi, protozoa, viruses, and algae, wherein the composition is in the form of an aggregate, and wherein the aggregate comprises a polymer binder.

34. The composition of claim 33, wherein the composition comprises more than 10.01% by weight of the insoluble rare earth-containing compound.

35. The composition of claim 30, wherein the biological contaminant comprises one or more of bacteria, fungi, protozoa, viruses, and algae, wherein the water insoluble rare earth-containing compound is at least one of incorporated into and coated onto a substrate, and wherein the substrate is at least one of sintered ceramics, sintered metals, microporous carbon, glass fibers, and cellulosic fibers.

36. The composition of claim 35, wherein substrate is a woven material in the form of a porous membrane, filter, or other fluid permeable structure.

37. The composition of claim 35, wherein the substrate is one of a mesh, screen, tube, honeycombed structure, monolith, and block.

38. The composition of claim 30, wherein the biological contaminant comprises one or more of bacteria, fungi, protozoa, viruses, and algae, wherein the water insoluble rare earth-containing compound is at least one of incorporated into and coated onto a filter block or monolith.

39. The composition of claim 33, wherein the biological contaminant comprises one or more of bacteria, fungi, protozoa, viruses, and algae, wherein the aggregate is disposed in a bed that provides a fluid permeable barrier to the aggregate.
